(19) United States
(12) Patent Application Publication
(10) Pub. No.: US 2012/0024326 A1
(43) Pub. Date: Feb. 2, 2012

(54) "DisNfect" Self contained and portable grocery cart disinfecting system

(76) Inventor: Robert Glenn McCarthy, Warrenton, VA (US)

(21) Appl. No.: 12/804,888

(22) Filed: Aug. 2, 2010

(51) Int. Cl. B08B 3/00

(52) U.S. Cl. 134/123

(57) ABSTRACT

A Grocery Cart Disinfecting System for any shopping carts. It was designed to help prevent the spread of germs, flu, H1N1, viruses, E.coli and salmonella.

The DisNfect Systems are rechargeable and totally portable, so there are no cords or hoses to hook up that limits where the unit can be used. The disinfect solution can be provided by a chemical supplier or company. The DisNfect System can do 1200 Grocery carts for less than .35 cents and disinfects the entire cart with its overhead system, compared to the expense of hand wipes and other disinfecting cleaners used by customers and staff.

The external hand held spraying apparatus can spray and cover up to 15,000 sqft. With a 4 gallon tank mounted on the inside or outside of the unit.

### BACKGROUND OF THE INVENTION

The invention was built on the premise of covering the entire shopping cart with a disinfectant to help prevent the spread of germs. This adds one more process to the shopping cart attendant daily routine. The rule for a cart attendants is not to push no more than 15 or 20 carts at a time, before the attendant brings the grocery carts into the store from the outside he or she lines them up, placing each cart inside of each other before entering the building the attendant rolls the DisNfect system over the carts by covering each one with an disinfectant leaving them germ free. After air drying for less than 10 minutes the attendant brings the carts into the store and repeats the process.

### SUMMARY OF THE INVENTION

The DisNfect system is built out of raw materials and metals. The DisNfect system is a totally portable and is a rechargeable unit. A disinfectant is mixed with the recommend parts of water in the 4 gallon tank. A pump then adds pressure through a chemical resistant hose to the overhead manifold or external spray gun that sprays a mist on the grocery carts. The mist is created with low pressure atomizing spray tips. This is all powered by a 12 volt wheel chair battery that is rechargeable.

### BRIEF DESCRIPTION OF THE DRAWING

FIG. 1 is a perspective view of the ‘DIS N FECT” unit how the operator views (side view) the present invention.

FIG. 2 is a rear view in where after the cart is disinfected the process is over.

FIG. 3 is an over view of the front where the carts would be entering to be disinfected.

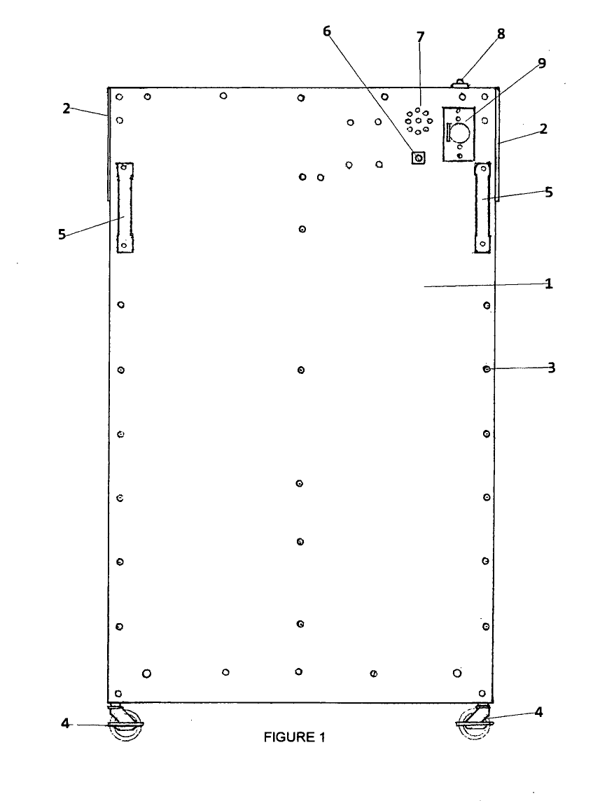

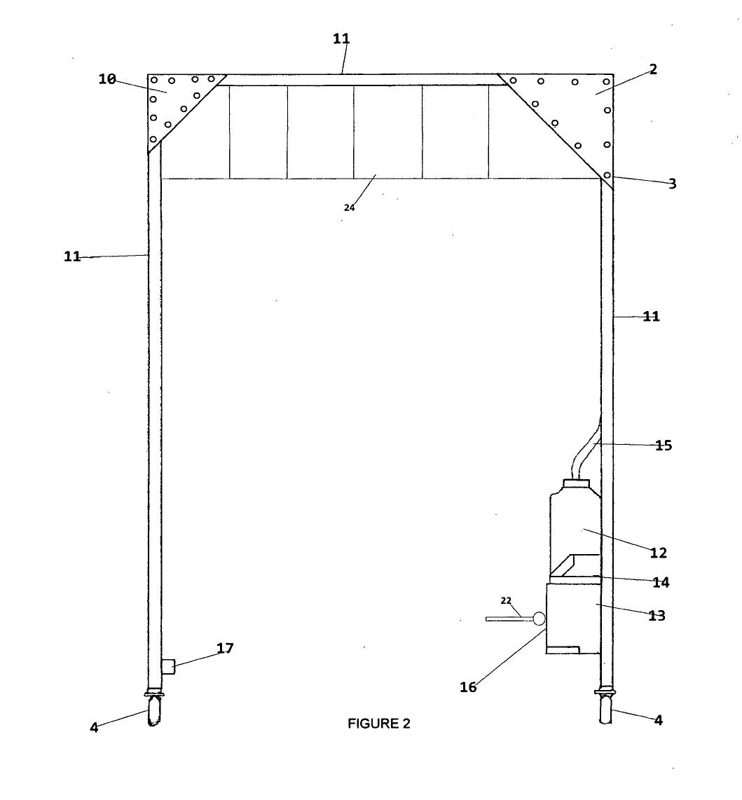

There are four sides and a top to this invention. The fourth side is a mirror image to FIG. 1 and just a flat panel.

While only the preferred form of the invention has been the preferred form of the present invention is illustrated in the accompanying drawings, in which:

Shown here, it should be understood that various changes and modifications may be made within the scope of the claims attached hereto without departing from the spirit of the invention.

### DESCRIPTION OF THE PREFERRED EMBODIMENT

In FIG. 1 is the operator view “side view” the exterior sheathing.
1) is 1/16 inch flat aluminum.
2) ⅛ in flat aluminum cut in a 45 degree angle for bracing.
3) ½ and ¾ self tapping pan head screws.
4) 3 inch wheels with a 360 degree swivel capability and a lock to prevent movement.

The wheels are rated at 150 lbs per wheel.
5) Two handles so the operator can control the machine while in operation.
6) On and off switch.
7) Air vent for the pump not to over heat.

There are 3 vents total that are not shown.
8) Lanyard kill switch with U shape clip added for safety.

If the machine is left unattended the clip is detached so only the operator can operate the machine.
9) An inverted male outlet to connect to the 12 volt battery charger daily.

FIG. 2 “the rear view”
10) 6½ inch ⅛ flat aluminum corner brace.
11) 1 inch aluminum square tubing frame that is not welded only held together by its exterior sheathing and corner braces.
12) 2.5 gallon F style container with lid is used for disinfected chemical tank.
13) Rechargeable 12 volt lawn mower battery.
14) Water resistant battery case with lid.
15) ⅜ hose and connector to run the pump and manifold.
16) Aluminum shelf to hold the battery and tank.
17) 1 inch aluminum square tubing and shelf help to guide the carts thru machine.
22) Optional automatic cart switch.
24) Garage door or freezer see thru flap to cut down wind and contain disinfected.

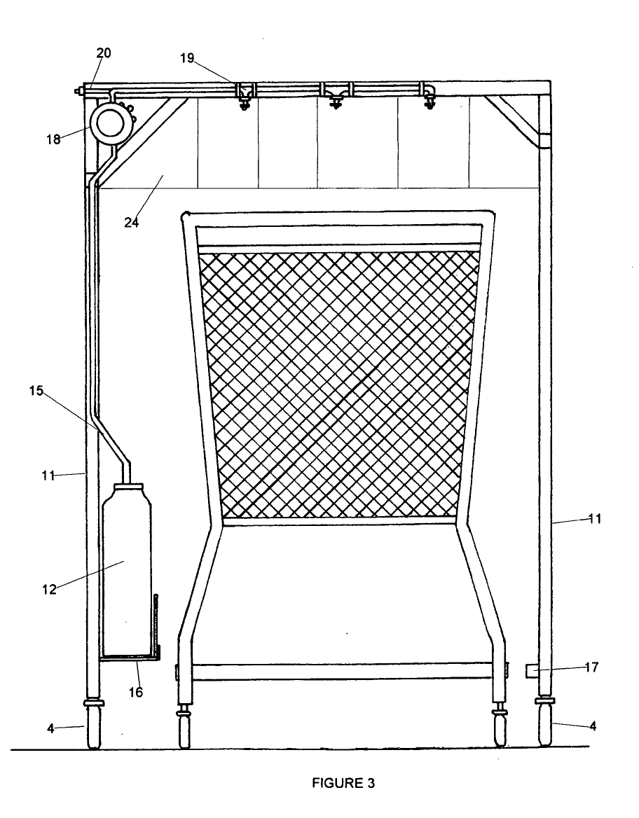

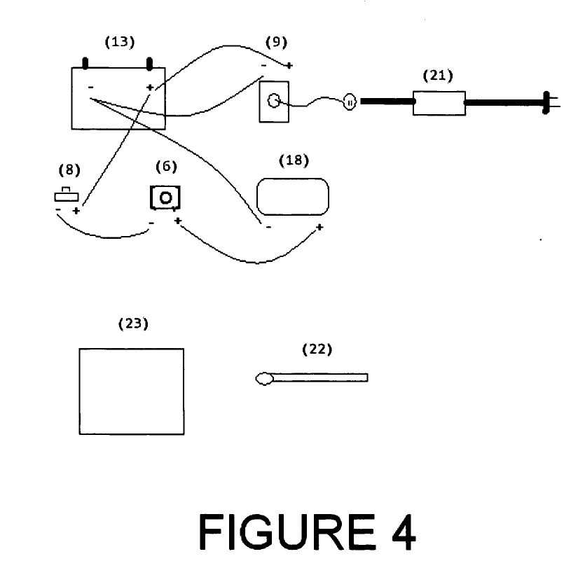

FIG. 3 “front view”
18) 12 volt pump with 45 psi/chemical resistant.
19) ¼ inch threaded pipe to make manifold with 3 atomizing spray heads to produce a misting affect.
20) ¼ inch threaded pipe to make manifold with two or three pressure tips to rinse cart if desired by adding a hose or pressure washer this process the carts are pushed thru the machine.

FIG. 4 “wiring diagram”
13) 12 volt wheel chair rechargeable battery.
9) 2 plug wiring connection for battery charger.
21) a slow charging 12 volt charger that is plug into an electrical outlet and plug into the machine to charge battery.
6) On and off switch.
8) Optional kill switch.
18) 12 volt 45 psi pump/chemical resistant.
23) Optional solar power to charge battery.
22) Optional automatic cart switch.

I claim:

1. Disinfecting apparatus for grocery carts and the like comprising:
Grocery Cart Disinfecting System,
Portable frame,
Self contained power,
No cord or hoses, Rechargeable,
4 gallon tank to disinfect 1200 grocery carts,
Frame is on wheels, light weight and small enough to bring into store and recharged at night,
Made of aluminum for added strength and durability,
Frame is cover by plastic paint to cut down the glare of the machine,
Grocery store attendants can operate easily and maintain the machine by just charging and adding disinfectant when needed,
Disinfecting apparatus with atomizing spray heads cause a wind tunnel affect to completely covering the whole surface of the cart, not just the handle,
The machine can roll over multiple carts and air dry in minutes
The disinfecting machine helps the spread of germs,
Disinfect daily and after each use.
